(19) United States
(12) Patent Application Publication
(10) Pub. No.: US 2022/0299494 A1
(43) Pub. Date: Sep. 22, 2022

(54) SYSTEMS AND METHODS FOR HIGH-THROUGHPUT SCREENING AND ANALYSIS OF DRUG DELIVERY SYSTEMS IN VITRO

(71) Applicant: Trustees of Tufts College, Medford, MA (US)

(72) Inventors: David L. Kaplan, Concord, MA (US); Qiaobing Xu, Lexington, MA (US)

(21) Appl. No.: 17/753,312

(22) PCT Filed: Aug. 28, 2020
(86) PCT No.: PCT/US20/48626

§ 371 Date: Feb. 28, 2022

Related U.S. Application Data
(60) Provisional application No. 62/892,945, filed on Aug. 28, 2019.

(51) Int. Cl. G01N 33/15; C12N 5/071; A61K 9/51; G01N 33/50

(52) U.S. Cl. CPC G01N 33/15; C12N 5/0697; A61K 9/5123; G01N 33/5082; G01N 33/502

(57) ABSTRACT

The present disclosure provides a method for screening drug delivery vehicles for use in delivering cargo via oral delivery. The method includes introducing a drug delivery vehicle comprising an imaging agent into a lumen of an artificial intestine system composed of a scaffold matrix material. The scaffold matrix material includes an interconnected network of pores, intestinal epithelial cells positioned on an inner surface of the lumen, and human-based cells positioned within the pores and surrounding the intestinal epithelial cells. The method includes maintaining the artificial intestine system in physiologically relevant conditions for a predetermined length of time, and detecting a color change induced by the imaging agent within at least a portion of the human-based cells.

### CROSS-REFERENCE TO RELATED APPLICATIONS

This application claims the benefit of priority to U.S. Provisional Application Ser. No. 62/892,945 filed on Aug. 28, 2019, the entire contents of which are incorporated by reference herein.

### STATEMENT REGARDING FEDERALLY SPONSORED RESEARCH

This invention was made with government support under grant P41EB002520 and EB027170-01 awarded by the National Institutes of Health. The government has certain rights in the invention.

### BACKGROUND

The administration of most current FDA approved protein and nucleic acid based drugs is involved with a needle. Oral delivery is considered an option to avoid the discomfort and pain associated with needle-involved injections, resulting in better patient acceptance. However, the harsh conditions in the GI tract such as low pH, enzymatic degradation and the inefficient crossing the intestinal epithelial layer hinder the oral route for the delivery of many biologics based drugs, including proteins and nucleic acids.

Many oral delivery systems have been developed to enhance the bioavailability of drugs. In particular, lipid-based carriers such as liposomes, solid lipid nanoparticles (SLNs), and nanostructured lipid nanoparticles, have received increasing attention for oral drug delivery. These lipid nanoparticles showed some success for orally delivering small molecule drugs, peptides and proteins in animal models. Nevertheless, insight into the in vivo biological fate of lipid nanoparticles after crossing the intestinal epithelial layer remains limited.

Several studies showed that the lipid nanoparticles can be taken up or transported across the GI tract as intact nanoparticles after oral administration. However, it is unclear whether the lipid nanoparticles enter into circulation as intact nanoparticles after absorption. For the delivery of nucleic acids or gene-editing proteins with an intracellular function, maintaining the intact structure of the nanocomplexes after intestinal absorption is crucial for delivering the desired cargo into the targeted tissue and cells.

Animal models have been extensively utilized to assess in vivo oral drug delivery using nanoparticles. However, these models often lack relevance to human physiological conditions, thus hindering the use of animal models to accurately predict the behavior of nanoparticles. Many drugs have been tested successfully in animal studies but unfortunately failed in human trials.

### SUMMARY OF THE DISCLOSURE

The present disclosure provides, among other things, a method for screening drug delivery vehicles for use in delivery cargo via oral delivery. The method includes introducing a drug delivery vehicle comprising an imaging agent into a lumen of an artificial intestine system composed of a scaffold matrix material. In some aspects, the scaffold matrix material comprises an interconnected network of pores, intestinal epithelial cells intestinal epithelial cells positioned on an inner surface of the lumen, and human-based cells positioned within the pores and surrounding the intestinal epithelial cells. The method further includes maintaining the artificial intestine system in physiologically relevant conditions for a predetermined length of time, and detecting a color change induced by the imaging agent within at least a portion of the human-based cells.

In some aspects, the method further includes quantifying the number of human-based cells in the artificial intestine system that undergo the color change. The method may be repeated for a plurality of different drug delivery vehicles, where the quantity of human-based cells that undergo a color change may be compared for each of the drug delivery vehicles to screen and/or compare the performance of the vehicles. The method provides an efficient, high-throughput way to screen and analyze drug delivery systems in vitro.

In some aspects, the method further includes generating one or more report that includes (i) a list ranking a portion or all of the drug delivery vehicles based on a quantity of human-based cells in the artificial intestine system that experience the color change, and/or (ii) a graph plotting the quantity of human-based cells in the artificial intestine system that experience the color change for at least a portion of the different drug delivery vehicles, and/or (iii) an identification of the drug delivery vehicle with the highest quantity of human-based cells that experience the color change.

In some aspects, the drug delivery vehicle includes a lipid nanoparticle.

In some aspects, the imaging agent includes a fluorescent compound. In some aspects, the imaging agent includes a gene-editing agent. The gene-editing agent may activate fluorescence within the human-based cells.

In some aspects, the human-based cells include an adenocarcinoma-based cell including, but not limited to, HeLa cells. In some aspects, the HeLa cells that include a fluorescent compound that is activated by the gene-editing agent in the drug delivery vehicle.

In some aspects, the intestinal epithelial cells include an adenocarcinoma-based cell including, but not limited to, CaCO-2 cells, HT29-MTX cells, and combinations thereof.

In some aspects, the intestinal epithelial cells include at least one of enterocytes, fibroblasts, Goblet cells, Paneth cells, and enteroendocrine cells.

In some aspects, the scaffold matrix material is composed of a biologically-based polymer, such as silk fibroin.

In some aspects, prior to introducing the drug delivery vehicles to the artificial intestine system, the drug delivery vehicles are pre-screened in a two-dimensional culture system. For example, the method includes introducing the drug delivery vehicle into an upper plate of a two-dimensional culture system having (i) a lower plate having human-based cells positioned on a surface of the lower plate, and (ii) an upper plate comprising a porous membrane and intestinal epithelial cells positioned on a surface of the porous membrane, where the upper plate is separated from the lower plate by a distance, where the upper plate is spaced from the lower plate by a distance. The method further includes maintaining the two-dimensional culture system in physiologically relevant conditions for a predetermined length of time, and detecting a color change induced by the imaging agent within at least a portion of the human-based cells.

In some aspects, the method includes quantifying the number of human-based cells in the two-dimensional culture system that undergo the color change. The method may include repeating the aforementioned steps in the two-dimensional culture system for a plurality of drug delivery vehicles, and selecting at least a portion of the delivery vehicles based on the quantity of human-based cells in the two-dimensional culture system that undergo the color change, and perform the any one of the aforementioned method steps for the artificial intestine system.

In some aspects, the present disclosure provides a system for screening drug delivery vehicles. The artificial intestine system is composed of a scaffold matrix material having (i) an interconnected network of pores, (ii) a lumen extending through the scaffold matrix material, (iii) a first region of cells, the first region of cells comprising intestinal epithelial cells positioned on an inner surface of the lumen, and (iv) a second region of cells, the second region of cells comprising human-based cells positioned within the pores and surrounding the intestinal epithelial cells.

In some aspects, the first region of cells forms a monolayer of cells positioned on the inner surface of the lumen.

In some aspects, the second region of cells express a fluorescent compound when expose to an enzyme recombinase that induces enzyme-mediated gene recombination. In some aspects, the second region of cells completely surrounds the first region of cells. In some aspects, the second region of cells directly contacts the first region of cells in the scaffold matrix material.

In some aspects, the second region of cells has a thickness that is at least 1.1 times greater than the first region of cells, or at least 2 times greater, at least 3 times greater, at least 4 times greater, or at least 5 times greater, or at least 6 times greater, or at least 7 times greater, or at least 8 times greater, or at least 9 times greater, to less than 10 times greater, or less than 15 times greater, or less than 20 times greater, or less than 50 times greater, or less than 100 times greater, or more.

In some aspects, the present disclosure provides a method for screening drug delivery vehicles for use in delivering cargo via oral delivery. The method includes introducing the drug delivery vehicle into an upper plate of a two-dimensional culture system having (i) a lower plate having human-based cells positioned on a surface of the lower plate, and (ii) an upper plate comprising a porous membrane and intestinal epithelial cells positioned on a surface of the porous membrane, wherein the upper plate is separated from the lower plate by a distance, wherein the upper plate is spaced from the lower plate by a distance. The method includes maintaining the two-dimensional culture system in physiologically relevant conditions for a predetermined length of time, and detecting a color change induced by the imaging agent within at least a portion of the human-based cells.

These and other advantages and features of the present invention will become more apparent from the following detailed description of the preferred embodiments of the present invention when viewed in conjunction with the accompanying drawings.

### DESCRIPTION OF THE DRAWINGS

FIG. 1 is a flow chart illustrating the steps of a method for screening drug delivery vehicles for use in delivering cargo via oral delivery in accordance with some embodiments of the present disclosure.

FIG. 2 is a schematic illustration of a drug delivery vehicle in the form of a lipid nanoparticle in accordance with some embodiments of the present disclosure.

FIG. 3 is a schematic illustration of an artificial intestine system in accordance with some embodiments of the present disclosure.

FIG. 4 is a schematic illustration of a two-dimensional culture system in accordance with some embodiments of the present disclosure.

FIG. 5 is an illustration showing the synthesis of cationic lipidoids conducted through ring-opening reactions between the lipophilic tails and various aliphatic amine heads in accordance with some embodiments of the present disclosure.

FIG. 6 is a graph illustrating cellular uptake of HeLa-DsRed cells treated with (−30)GPF-Cre alone or with different drug delivery vehicles in accordance with some embodiments of the present disclosure.

FIG. 7 is a graph illustrating DsRed expression level of HeLa-DsRed cells treated with (−30)GPF-Cre alone or with different drug delivery vehicles in accordance with some embodiments of the present disclosure.

FIG. 8 is a fluorescence image of the HeLa-DsRed cells seeded on the lower plate of the two-dimensional culture system after (−30)GFP-Cre proteins alone or different LNP/GFP-Cre complexes were added into the upper plate for 48 h in accordance with some embodiments of the present disclosure. Scale bar: 200 μm.

FIG. 9 is graph illustrating cellular uptake of HeLa-DsRed cells seeded on the lower plate of the two-dimensional culture system after (−30)GFP-Cre proteins alone or different LNP/GFP-Cre complexes were added into the upper plate for 48 h in accordance with some embodiments of the present disclosure.

FIG. 10 is a graph illustrating DsRed expression level of HeLa-DsRed cells seeded on the lower plate of the two-dimensional culture system after (−30)GFP-Cre proteins alone or different LNP/GFP-Cre complexes were added into the upper plate for 48 h in accordance with some embodiments of the present disclosure.

FIG. 11 is a graph illustrating TEER value change of the in the period of the epithelial monolayer in accordance with some embodiments of the present disclosure.

FIG. 12(a-f) illustrate CLSM images of immunostaining of the two-dimensional culture system after treatment with different drug delivery vehicles in accordance with some embodiments of the present disclosure. FIG. 12a illustrates a control; FIG. 12b illustrates (−30)GFP-Cre proteins alone; FIG. 12c illustrates LNP/GFP-Cre complexes treatment for 1 h, FIG. 12d illustrates LNP/GFP-Cre complexes treatment for 6 h; FIG. 12e illustrates LNP/GFP-Cre complexes treatment for 12 h; and FIG. 12f illustrates LNP/GFP-Cre complexes treatment for 24 h, respectively. Scale bar: 15 μm.

FIG. 13 is a series of CLSM images of the scaffolds after treatment with (−30)GFP-Cre alone or different concentrations of complexes. The images were captured at the surface of the lumen and the bulk space of the 3D scaffolds, respectively, in accordance with some embodiments of the present disclosure. Scale bar: 200 μm.

FIG. 14 is a graph illustrating the hydrodynamic diameter of drug delivery vehicles (e.g., LNPs) and LNP/GFP-Cre complexes in accordance with some embodiments of the present disclosure.

FIG. 15 is a graph illustrating the polydispersity of drug delivery vehicles (e.g., LNPs) and LNP/GFP-Cre complexes in accordance with some embodiments of the present disclosure.

### DETAILED DESCRIPTION

Currently, conventional techniques for screening oral drug delivery compounds include using in vivo animal models. However, animal models lack relevance to human physiological conditions, thus hindering the use of animal models to accurately predict the behavior of the drug delivery compound.

In contrast, various embodiments of the present disclosure provide systems and methods for screening drug delivery vehicles for use in delivering cargo via oral delivery. In some embodiments, the systems and methods described herein are relatively simple to perform, and more closely resemble the complex microenvironment found in vivo, and thus can more accurately reflect human outcomes. The systems and methods provided herein allow researchers to screen and identify drug delivery vehicles that are capable of crossing the epithelial layer of a patient's intestine in order to transport the cargo to a region of interest of the subject.

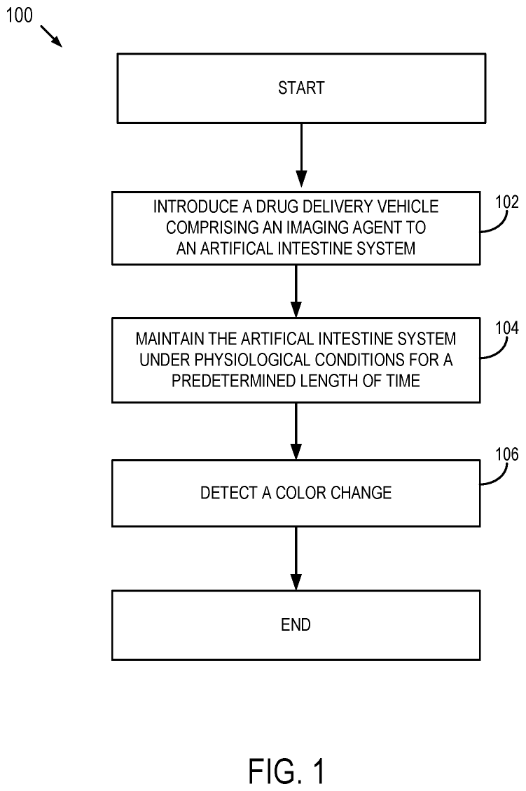

Referring to FIG. 1, the present disclosure provides a method 100 of screening drug delivery vehicles for use in delivering one or more imaging agent to a region of interest in a subject (e.g., intestine) via oral delivery. As used herein, the term “drug delivery vehicle” may refer to molecular cages that are sized to encapsulate one or more imaging and/or therapeutic agent. In some embodiments, the drug delivery vehicles are non-toxic, biocompatible, non-immunogenic, and biodegradable. Suitable drug delivery vehicles include, but are not limited to, liposomes, polymeric micelles, lipid nanoparticles, dendrimers, biodegradable particles, DNA nanostructures, and combinations thereof.

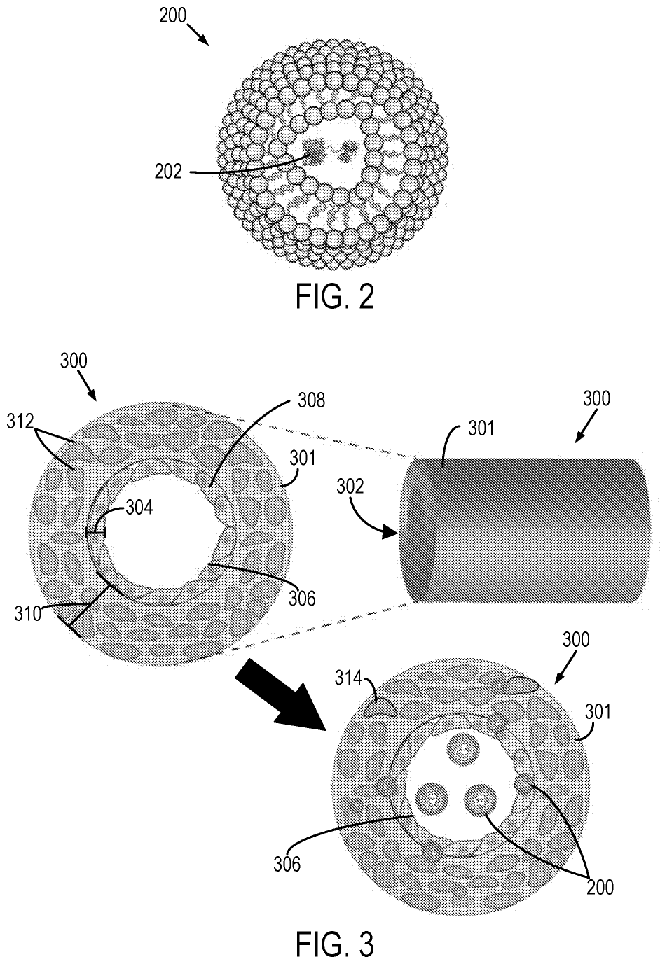

As used herein, the term “imaging agent” may refer to a compound and/or chemical moiety that facilitates differentiating cells during live cell imaging. Exemplary imaging agents include, but are not limited to, fluorescent compounds, quantum dots, dyes and cell stains. In some embodiments, the imaging agent refers to compounds and/or chemical moieties capable of inducing a color change (e.g., visible color change and/or fluorescence) inside of a cell. In one non-limiting example, the imaging agent is a gene-editing agent. For example, the gene-editing agent may be an enzyme capable of inducing enzyme-mediate gene recombination that promotes fluorescence within cells in a region of interest (e.g., Cre-mediated gene recombination inducing red fluorescence of DsRed in a HeLa-DsRed cell line). In some embodiments, the imaging agent includes a combination of one or more florescent compound, quantum dot, dye, cell stain, and/or enzyme capable of inducing enzyme-mediated gene recombination. FIG. 2 illustrates an exemplary drug delivery vehicle 200 comprising an imaging agent 202 in accordance with some embodiments of the present disclosure.

Referring back to FIG. 1, the method 100 further includes introducing the drug delivery vehicle 200 and imaging agent 202 into a lumen 302 of an artificial intestine system 300. FIG. 3 illustrates an exemplary artificial intestine system 300 in accordance with some embodiments of the present disclosure. In some embodiments, the artificial intestine system 300 is composed of a scaffold matrix material 301 having pores.

In some embodiments, the scaffold matrix material 301 is composed of one or more biologically-compatible polymer. Suitable biologically-compatible polymers include silk fibroin, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, and polyanhydrides.

In some embodiments, the silk fibroin is derived from Bombyx mori silkworm cocoons, is a biocompatible and biodegradable material that degrades slowly in the body, is readily modified into a variety of formats, and generates mechanically robust materials.

As used herein, the term “fibroin” includes, but is not limited to, silkworm fibroin and insect or spider silk protein. In some embodiments, fibroin is obtained from a solution containing a dissolved silkworm silk or spider silk. In some embodiments silkworm silk protein is obtained, for example, from Bombyx mori, and spider silk is obtained from Nephila clavipes. In some embodiments, silk proteins suitable for use in the present invention may be obtained from a solution containing a genetically engineered silk, such as from bacteria, yeast, mammalian cells, transgenic animals or transgenic plants.

In some embodiments, silk fibroin scaffolds comprising silk fibroin may be made using one or more silk solutions, which are known to be highly customizable and allow for the production of any of a variety of end products. As such, in some embodiments, scaffold matrix materials 301 may be produced using any of a variety of silk solutions. Preparation of silk fibroin solutions has been described previously, e.g., in WO 2007/016524, which is incorporated herein by reference in its entirety. The reference describes not only the preparation of aqueous silk fibroin solutions, but also such solutions in conjunction with bioactive agents.

In accordance with various embodiments, a silk solution may comprise any of a variety of concentrations of silk fibroin. In some embodiments, a silk solution may comprise 0.1 to 30% by weight silk fibroin. In some embodiments, a silk solution may comprise between about 0.5% and 30% (e.g., 0.5% to 25%, 0.5% to 20%, 0.5% to 15%, 0.5% to 10%, 0.5% to 5%, 0.5%) to 1.0%) by weight silk fibroin, inclusive. In some embodiments, a silk solution may comprise at least 0.1% (e.g., at least 0.5%, 1%, 2%, 3%, 4%, 5%, 6%, 7%, 8%, 9%, 10%, 15%, 20%), 25%)) by weight silk fibroin. In some embodiments, a silk solution may comprise at most 30% (e.g., at most 25%, 20%, 15%, 14%, 13%, 12% 11%, 10%, 5%, 4%, 3%, 2%, 1%) by weight silk fibroin.

In accordance with various embodiments, the scaffold matrix material 301 disclosed herein can comprise any amount/ratio of silk fibroin to the total volume/weight of the overall scaffold. In some embodiments, the amount of silk fibroin in the solution used for making a provided silk fibroin composition itself can be varied to vary properties of the end silk fibroin composition. By way of specific example, in some embodiments, silk fibroin comprises at least 1% of a provided composition by weight (e.g., at least 2%, 3%, 4%, 5%, 6%, 7%, 8%, 9% 10%, 15%, 20%), 25%) or more). In some embodiments, silk fibroin comprises at most 35% of a provided composition by weight (e.g., at most 30%, 25%, 20%, 15%, 10%, 5% or less). In some embodiments, silk fibroin comprises between 1-35% of a provided composition by weight (e.g., between 1-30%, 1-25%, 1-20%, 1-15%, 1-10%, 1-5%, 5-25%, 5-20%, 5-15%, 5-10%). In some embodiments, silk fibroin comprises 4-5% silk fibroin by weight.

In accordance with various embodiments, silk used in provided methods and systems is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some embodiments, the silk cocoons are boiled for at least 60 minutes, at least 70 minutes, at least 80 minutes, at least 90 minutes, at least 100 minutes, at least 110 minutes, at least 120 minutes, or longer. Additionally or alternatively, in some embodiments, silk cocoons can be heated or boiled at an elevated temperature. For example, in some embodiments, silk cocoons can be heated or boiled at about 100° C., 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher.

In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, the aqueous solution used in the process of degumming silk cocoons comprises about 0.02M Na2C03. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

In some embodiments, the silk fibroin is substantially depleted of its native sericin content (e.g., 5% (w/w) or less residual sericin in the final extracted silk). In some embodiments, the silk fibroin is entirely free of its native sericin content. As used herein, the term “entirely free” (i.e. “consisting of terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of) means that only trace amounts of the substance can be detected, is present in an amount that is below detection, or is absent. If necessary, the silk solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution.

In some embodiments, a silk fibroin solution can optionally, at a suitable point, be filtered and/or centrifuged. For example, in some embodiments, a silk fibroin solution can optionally be filtered and/or centrifuged following the heating or boiling step. In some embodiments, a silk fibroin solution can optionally be filtered and/or centrifuged following the dialysis step. In some embodiments, a silk fibroin solution can optionally be filtered and/or centrifuged following the step of adjusting concentrations. In some embodiments, a silk fibroin solution can optionally be filtered and/or centrifuged following the step of reconstitution. In any of such embodiments, the filtration and/or centrifugation step(s) can be carried out to remove insoluble materials. In any of such embodiments, the filtration and/or centrifugation step(s) can be carried out to selectively enrich silk fibroin fragments of certain molecular weight(s).

In some embodiments, pores in the scaffold matrix material 301 have a diameter suitable for seeding one or more human-based cell. In some embodiments pores in the scaffold matrix material 301 have a diameter between about 1-1,000 μm, (e.g., between about 1-100, 20-100, 30-100, 40-100, 50-100, 60-100, 70-100, 80-100, 90-100, 50-1,000, 100-1,000, 200-1,000, 300-1,000, 400-1,000, 500-1,000, 600-1,000, 700-1,000, 800-1,000, or 900-1,000 μm) inclusive. In some embodiments, pores have a diameter between about 100-1,000 μm, inclusive. In some embodiments, pores have a diameter between about 100-300 μm, inclusive. In some embodiments, pores have a diameter between about 150-250 μm, inclusive.

In some embodiments, the scaffold matrix material 301 may be of a variety of different thicknesses. In some embodiments, the scaffold matrix material 301 is less than or equal to 100 cm thick. In some embodiments, a silk scaffold is between 0.1 and 100 cm thick (e.g., 0.2-100, 0.5-10, 0.2-9, 0.2-8, 0.2-7, 0.2-6, 0.2-5, 0.2-4, 0.2-3, 0.2-2, 0.2-1, 0.5-1, 0.2-0.9, 0.2-0.8, 0.2-0.7, 0.2-0.6, 0.2-0.5, 0.2-0.4, 0.2-0.3 cm thick). In some embodiments, scaffold matrix material 301 is about 0.2-0.5 μm thick, inclusive. In some embodiments, the scaffold matrix material 301 is of a substantially uniform thickness. In some embodiments, the silk scaffold varies in thickness across a particular length (e.g., a 1 cm).

Referring back to FIG. 3, in some embodiments, the scaffold matrix material 301 includes a first region of cells 304 positioned on an inner surface 306 of the lumen 302. In some embodiments, the first region of cells 304 comprises intestinal epithelial cells 308. The intestinal epithelial cells 308 may be positioned in the pores of the scaffold matrix material 301 located along the inner surface 306 of the lumen 302. In some embodiments, the first layer of cells 304 forms a monolayer of cells. In some embodiments, the first region of cells 304 is formed of multiple layers of intestinal epithelial cells, e.g., at least 2 layers, at least 3 layers, at least 4 layers, or at least 5 layers, or more.

In some embodiments, the intestinal epithelial cells in the first region of cells 304 comprises an intestinal-based immortalized cell line or an intestinal-based adenocarcinoma cell. Exemplary adenocarcinoma cells include, but are not limited to, CaCO-2 cells, HT-29 cells, HT29-MTX cells, and combinations thereof.

In some embodiments, the intestinal epithelial cells in the first region of cells 304 creates an in vivo intestine-like composition. In some embodiments, the intestinal epithelial cells in the first region of cells 304 includes one or more of enterocytes, Goblet cells, Paneth cells, and enteroendocrine cells. In some embodiments, intestinal epithelial layer includes multipotent stem cells (i.e., cells capable of differentiating into enterocytes, Goblet cells, Paneth cells, and/or enteroendocrine cells).

In some embodiments, the intestinal epithelial cells in the first region of cells 304 includes one or more digestive enzymes. By way of specific example, in some embodiments, the digestive enzyme secretion is or comprises secretion of one or more of alkaline phosphatase, secretin, cholecystokinin, maltase, lactase, gastric inhibitory peptide, motilin, somatostatin, erepsin, and sucrase.

In some embodiments, the intestinal epithelial cells in the first region of cells 304 includes nervous system cells. In some embodiments, the nervous system cells are human nervous system cells. In some embodiments, the nervous system cells are or comprise afferent nerve cells. In some embodiments, the nervous system cells are or comprise efferent nerve cells. In some embodiments, the nervous system cells comprise glial cells. In some embodiments, at least some of the plurality of nervous system cells provide functional innervation to at least some of the enterocytes, Paneth cells, enteroendocrine cells, and/or Goblet cells.

In some embodiments, the nervous system cells comprise neuronal nitric oxide synthase (nNOS)-expressing neurons. In some embodiments, the intestinal epithelial cells in the first region of cells 304 are capable of initiating an antimicrobial response (e.g., in response to a microbe or portion thereof). In some embodiments, an antimicrobial response is or comprises upregulated gene and/or protein expression of one or more of lymphocyte antigen 96 (LY96), toll-like receptor-2 (TLR2), toll-like receptor-4 (TLR4), toll-like receptor-5 (TLR5), toll-like receptor-6 (TLR6), c-reactive protein (CRP), deleted in malignant brain tumors-1 (DMBT1), interferon regulatory factor-7 (IRF7), z-DNA-binding protein 1 (ZBP1), chemokine (C-C motif) ligand 3 (CCL3), C—X—C motif chemokine 1 (CXCL1), C—X—C motif chemokine 2 (CXCL2), interleukin-12 subunit alpha (IL12A), interleukin-12 subunit beta (IL12B), interleukin 1 beta (ILIB), interleukin 6 (IL6), myeloid differentiation primary response gene 88 (MYD88), nucleotide-binding oligomerization domain-containing protein 1 (NODI), nucleotide-binding oligomerization domain-containing protein 2 (NOD2), Ras-related C3 botulinum toxin substrate 1 (RAC 1), p65 (RELA), tumor necrosis factor (TNF), bactericidal permeability-increasing protein (BPI), cathelicidin (CAMP), cathepsin G (CTSG), lysozyme (LYZ), myeloperoxidase (MPO), secretory leukocyte protease inhibitor (SLPI), mitogen-activated protein kinase kinase 1 (MAP2K1), mitogen-activated protein kinase 1 (MAPK1), mitogen-activated protein kinase 8 (MAPK8), JUN, killer cell immunoglobulin-like receptor subunit a (NKB 1A), caspace 1 (CASP1), and apoptosis-associated speck-like protein containing a CARD (PYCARD).

Referring back to FIG. 3, in some embodiments, the scaffold matrix material 301 includes a second region of cells 310 comprising human-based cells 312. In some embodiments, the human-based cells 3012 comprise an immortalized cell line or an adenocarcinoma-based cell. Exemplary immortalized cells include, but are not limited to, HeLa cells. In some embodiments, the human-based cells 312 may express a fluorescent protein upon enzyme-mediated gene recombination (i.e., when the drug delivery vehicle delivers the imaging agent 202 to the second region of cells 310). Exemplary immortalized cells that express fluorescent proteins include, but are not limited to, HeLa-DsRed cells.

Referring back to FIG. 1, the method 100 further includes maintaining the artificial intestine system in physiologically relevant conditions for a predetermined length of time, as indicated by process step 104. During the predetermined length of time the drug delivery vehicle 200 may pass through tight junctions in the first region of cells 304, and pass into the second region of cells 310. The drug delivery vehicle 200 may then deliver the imaging agent 202 to the cells in the second region of cells 310.

As used herein, “physiologically relevant conditions” may refer to a range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40° C., about 30-40° C., about 35-40° C., about 37° C., and atmospheric pressure of about 1. In some embodiments, physiological conditions utilize or include an aqueous environment (e.g., water, saline, Ringers solution, or other buffered solution); in some such embodiments, the aqueous environment is or comprises a phosphate buffered solution (e.g., phosphate-buffered saline).

In some embodiments, the predetermined length of time ranges from 30 minutes to one week. In some embodiments, the predetermined length of time is at least 30 minutes, or at least an hour, at least six hours, or at least 12 hours, or at least 24 hours, or at least two days, or at least three days, or at least four days, to less than five days, or less than six days, or less than a week.

Referring back to FIG. 1, the method 100 further includes detecting a color change 314 in the cells induced by the imaging agent within at least a portion of the cells within the second cell region 310. The color change 314 may be detected using various methods known to those skilled in the art. For example, the cells may be visually or optically inspected to track a visual color change 314, or various fluorescence detection devices, such as flow cytometry may be used to detect fluorescence within at least a portion of the cells. In some embodiments, the method 100 further includes quantifying the number cells within the second region of cells 310 that undergo a color change 314.

In some embodiments, the method 100 includes repeating steps 102-104 for a plurality of different drug delivery vehicles 200 having an imaging agent 202 therein. The method 100 may optionally further including screening, or otherwise determining, which drug delivery vehicle 200 performed the best at delivering the imaging agent to the second region of cells 310. In some embodiments, the drug delivery vehicles 200 may be screened by quantifying the number of human-based cells 312 in the second region of cells 310 that undergo a color change 314. The method 100 may further optionally include generating a report that ranks the different drug delivery vehicles 200 based on the quantity of human-based cells 312 in the artificial intestine system that experienced the color change 314, generating a plot of the quantity of human-based cells 312 in the artificial intestine system that experience the color change 314 for each of the different drug delivery vehicles 200, and/or identifying the best performing drug delivery vehicle 200 with the highest quantity of human-based cells 312 that experience the color change 314.

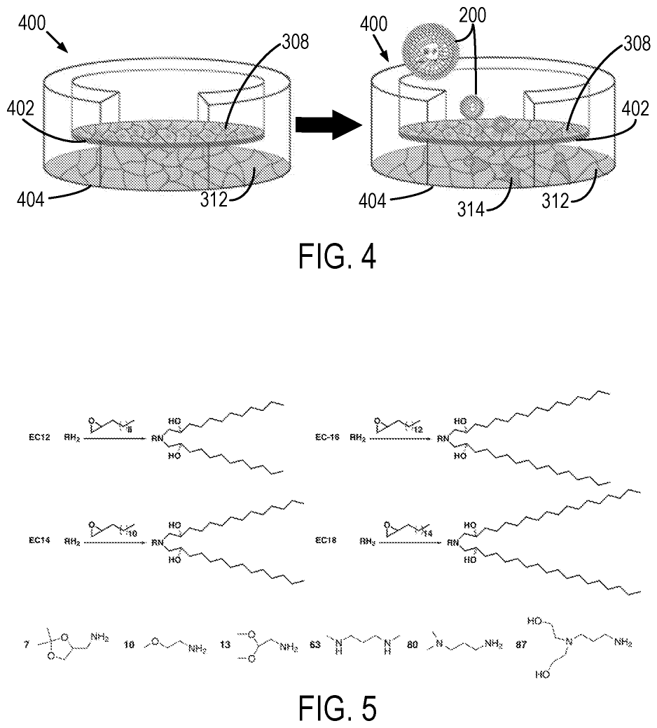

In some embodiments, prior to introducing the drug delivery vehicle 200 to the three-dimensional artificial intestine system 300, the drug delivery vehicle 200 may be optionally screened in a two-dimensional culture system 400. FIG. 4 illustrates an exemplary two-dimensional culture system 400 in accordance with some embodiments of the present disclosure. In some embodiments, the drug delivery vehicle 200 is screened in the two-dimensional culture system 400 by introducing the drug delivery vehicle 200 and imaging agent 200 in an upper plate 402 of the two-dimensional culture system 400. In some embodiments, the upper plate 402 includes a porous membrane and intestinal epithelial cells 308 positioned on a surface of the porous membrane. In some embodiments, the intestinal epithelial cells for a monolayer on the porous membrane. The intestinal epithelial cells 308 used in the two-dimensional plate may be the same as the intestinal epithelial cells 308 used in the artificial intestine system 300.

In some embodiments, the two-dimensional culture system 400 includes a lower plate 404 that is separated from the upper plate 402 by a distance. In some embodiments, the lower plate 404 includes human-based cells 312 positioned on a surface of the lower plate 404. The human-based cells 312 may be the same as the human-based cells 312 used in the artificial intestine system 300.

In some embodiments, the method of screening using the two-dimensional culture system 400 further includes maintaining the two-dimensional culture system 400 in physiologically relevant conditions for a predetermined length of time. During the predetermined length of time the drug delivery vehicle 200 may pass through tight junctions in the intestinal epithelial cells and porous membrane of the first plate, and pass down to the human-based cells on the second plate. The drug delivery vehicle 200 may then deliver the imaging agent 200 to the cells in the human-based cells 312 on the lower plate 404.

The method of screening using the two-dimensional culture system 400 further includes detecting a color change 314 induced by the imaging agent within at least a portion of the cells the human-based cells 312 on the lower plate 404. The color change 314 may be detected using various methods known to those skilled in the art. For example, the cells may be visibly or optically inspected to track a visual color change 314, or various fluorescence detection devices, such as flow cytometry may be used to detect fluorescence within at least a portion of the cells. In some embodiments, the method further includes quantifying the number cells the human-based cells 312 on the lower plate 404 that undergo a color change 314.

In some embodiments, the method of screening using the two-dimensional culture system 400 includes repeating the specified steps for a plurality of different drug delivery vehicles 200 having an imaging agent 202 therein. The method 100 may optionally further including screening, or otherwise determining, which drug delivery vehicle 200 performed the best at delivering the imaging agent to the human-based cells 312 on the lower plate 404 prior to screening the drug delivery vehicles 200 in the artificial intestine system 300. In some embodiments, the two-dimensional culture system 400 may be used alone as a method for screening drug delivery vehicles 200 (i.e., without using the artificial intestine system 300).

The methods described herein can be deployed as a rapid screening tool for drug delivery vehicles. This screening can involve a first group of drug delivery vehicles, which are selected based off properties and/or simulations and/or any reason understood to a skilled artisan. The first step in the screening can involve screening the vehicles using the two-dimensional system. Vehicles that successfully pass the initial screening on the two-dimensional system are advanced to screening on the three-dimensional system. The second step in the screening can involve screening the vehicles that passed the test with the two-dimensional system using the three-dimensional system. Vehicles that successfully pass this second screening in the three-dimensional system are advanced to screening in animal models or other advanced screening methods. This rapid screening tool can save significant cost by allowing only the most promising targets to be tested in the most complicated environments, such as in animal models or human studies.

### Examples

The following examples will enable one of skill in the art to more readily understand the principles thereof. The following examples are presented by way of illustration and are not meant to be limiting in any way.

### Formation of Lipid Nanoparticles:

The lipidoids were synthesized according to the following procedure. Briefly, hydrophilic tails (1,2-epoxydodecane, 1,2-epoxytetradecane, 1,2-epoxyhexadecane, and 1,2-epoxyoctadecane) and individual amine head groups were mixed in a 5 mL Teflon-lined glass screw-top vial at a molar ratio of 2.4:1 (epoxide:amine), followed by a reaction at 80° C. without solvent for 48 h. The mixtures were then cooled to room temperature and purified through the flash chromatography on silica gel. The LNPs were formulated by the lipidoid, cholesterol, 1,2-dioleoyl-sn-glycero-3-phosphoethanolamine (DOPE), and DSPE-PEG2K at a mass ratio of 16:4:1:4 in the ethanol and then added to the sodium acetate solution (pH 5.0, 25 mM). The mixture was dialyzed against the pure water for 4 h using Slide-A-Lyzer MINI Dialysis Device (Millipore, 3.5K MWCO, 0.1 ml).

### Expression and Purification of (−30)GFP-Cre Protein:

The plasmid harboring (−30)GFP-Cre protein was expressed in E. coli BL21 STAR (DE3)-competent cells (Life Technologies). The E. coli was incubated in LB broth containing 100 mg/ml ampicillin at 37° C. overnight. Afterwards, the culture was added with isopropyl β-d-1-thio-galactopyranoside (IPTG) and incubated at 20° C. overnight. Then, the cells were collected through centrifugation at 8,000 g and re-suspended in the lysis buffer ((20 mM Tris, 1M KCl, 20% glycerol, pH 8.0). The cells were lysed by sonication and then centrifuged at 8,000 g for 15 min. Next, the precipitate was incubated with nickel-NTA resin at 4° C. for 30 min to capture His-tagged (−30)GFP-Cre protein. The resin was then transferred to a 20 ml-gravity column (Bio-Rad) and washed with 25 mM wash buffer (20 mM imidazole, 20 mM Tris, 1M KCl, 20% glycerol, pH 8.0) for three times. Then, the column was washed with 20 ml elution buffer ((250 mM imidazole, 20 mM Tris, 1M KCl, 20% glycerol, pH 8.0) for five times. Finally, the purified protein was dialyzed against lysis buffer and then concentrated by an Amicon ultracentrifugal filter (Millipore; 100 KDa MWCO).

### Intracellular Delivery of LNP/GFP-Cre Complexes:

The HeLa-DsRed cells were plated at 48-well plate at a density of 40,000 cells/well and incubated at 37° C. overnight. The LNPs were incubated with (−30)GFP-Cre at a mass ratio of 10:8 at room temperature for 15 min, forming stable complexes. The complexes containing 0.8 μg (−30)GFP-Cre were then added into individual wells for 6 h and then harvest for intracellular green fluorescence through flow cytometer (BD FACS Calibur, BD Science, CA). Similarly, for analysis of the gene recombination efficiency, the cells were treated with complexes under the same condition for 24 h, and then harvest for analyzing intracellular DsRed fluorescence using flow cytometer.

### LNPs-Mediated (−30)GFP-Cre Protein Delivery in 2D Transwell Culture Model:

The CaCO2 and HT29-MTX cells (3:1) were planted on the Transwell membrane (Pore size: 0.4 μm; Costar Corp.) at a density of 2×10⁵ cells/cm² in the DEME culture medium containing 10% FBS and 10 μg/ml human transferrin until the CaCO2/HT29-MTX cells were cultured to 100% confluence where TEER values reached over 400 Ω/cm². The HeLa-DsRed cells were seeded on the bottom of the 24-well plates at a density of 4×10⁴ cells/well. Then the LNPs complexes containing 80 μg (−30)GFP-Cre protein were added into the upper chamber of the Transwell system for 48 h. The TEER value was measured using the Millicell ERS Voltohmmeter (Millpore) during the period of delivery. After 48 h, the HeLa-DsRed cells were collected and analyzed by the flow cytometer. The Transwell membrane was fixed with 4% paraformaldehyde (PFA, Santa Cruz), treated with 0.1% Triton X-100 in PBS solution, and then blocked with 5% BSA solution for 2 h. Afterwards, the membranes were stained with the anti-E-cadherin (2.5 μg/ml, Invitrogen) at 4° C. overnight and then treated with Alexa Fluo 594 goat-anti mouse secondary antibody for 1 h. Subsequently, the Transwell membranes were washed with PBS for three times and scanned using Leica SP2 confocal microscope (Leica Microsystems).

### LNPs-Mediated (−30)GFP-Cre Protein Delivery in 3D Intestinal Tissue Models:

The 3D tissue system and the seeding procedure with intestinal epithelial cells was prepared as follows. Briefly, the CaCO2 and HT29-MTX cells were co-incubated at the surface of the lumen scaffold, while the HeLa-DsRed cells were re-suspended in collagen gel and delivered into the bulk space of the 3D scaffolds for 10 days. Then the LNP/GFP-Cre complexes containing 100 μg (high concentration) or 60 μg (low concentration) (−30)GFP-Cre protein were added into individual 3D tissue systems and cultured for 48 h. Afterwards, the scaffolds were washed with PBS for 3 times and fixed with 4% PFA at 4° C. overnight. Subsequently, the scaffolds were cut into small pieces by a scissors and then scanned by Leica SP2 confocal microscopy (Leica Microsystems).

### Results:

In the present example, a series of lipidoid nanoparticles (LNPs) were synthesized, and demonstrated that cationic LNPs formed stable complexes with the gene-editing Cre recombinase ((−30)GFP-Cre) through electronic self-assembly. A 2D transwell system was used to screen a library of LNPs and the resulting LNPs were then further validated in a 3D tissue engineered intestinal model.

In the 2D Transwell systems, Caco2/HT29-MTX cells were seeded on the transwell membrane forming a compact cell monolayer, and HeLa-DsRed cells were plated in the bottom chamber, to serve as a model for determining whether the LNPs formed intact nanoparticles to exert the gene-editing function. It was observed that the LNPs efficiently delivered the (−30)GFP-Cre protein, penetrating the cell monolayer seeded on the Transwell membranes in the 2D culture model and activating the HeLa-DsRed cells by endocytosis. LNP loaded (−30)GFP-Cre protein (LNP/GFP-Cre) were then tested in the 3D tissue engineered system, in which Caco2/HT29-MTX cells were seeded on the surface of the lumen and the bulk space was incubated with the HeLa-DsRed cells. After delivering the LNP/GFP-Cre complexes into the 3D cell culture system, the complexes successfully penetrated the Caco2/HT29-MTX monolayer and reached the scaffold bulk space to realize gene-editing functions (FIG. 1).

LNP chemistries with encapsulated gene-editing proteins were screened with a combination of 2D Transwell and 3D scaffold tissue models. Some of the nanoparticles were found to maintain stable structures to penetrate these in vitro intestinal lumen mimics, suggestive towards successful screening of designs with potential to reach the circulatory system in vivo to generate gene-editing functions at targeted sites.

In addition, different proteins for delivery required distinct lipidoids formulations. Thus, to verify effective LPNs for (−30)GFP-Cre delivery, we screened a small library of 12 lipidoids. The library of the lipidoids was synthesized through the ring-opening reaction between the lipophilic tails and various aliphatic amine heads, where the lipophilic tails were 1,2-epoxydodecane (EC12), 1,2-epoxytetradecane (EC14), 1,2-epoxyhexadecane (EC-16), and 1,2-epoxyoctadecane (EC18), respectively. The lipidoids were named by the lipophilic tail and the amine head number (FIG. 5). The LNPs were subsequently obtained by formulation with cholesterol, 1,2-dioleoyl-sn-glycero-3-phosphoethanolamine (DOPE), and 1,2-distearoyl-sn-glycero-3-phosphoethanolamine (DSPE)-PEG2K.

Next, to evaluate the intracellular protein delivery efficiency, the Cre recombinase, which has been fused to a negatively charged GFP variant to generate green fluorescence spontaneously, was used as the model cargo to assess efficiency. The HeLa-DsRed cell line expressing the red fluorescence DsRed upon Cre-mediated gene recombination was used to determine the (−30)GFP-Cre delivery efficiency. Following LNPs-mediated (−30)GFP-Cre delivery into the HeLa-DsRed cells for 6 h, the percentage of GFP-positive cells was determined to evaluate the uptake efficiency of LNP/GFP-Cre complexes by flow cytometry.

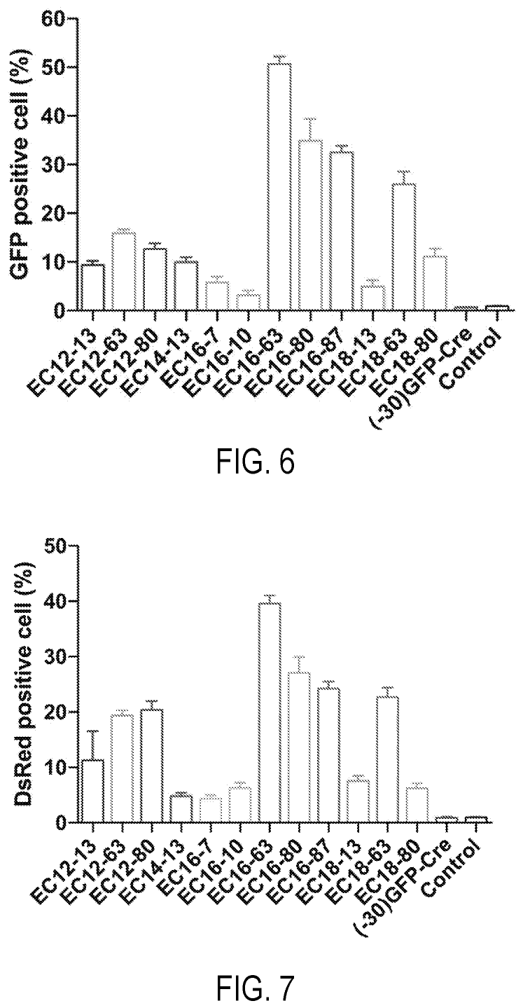

As shown in FIG. 6, negligible GFP-positive cells could be detected in the free (−30)GFP-Cre group, indicating that free GFP-Cre protein could not be taken up by the cells without the LNPs. In contrast, a significantly higher proportion of GFP-positive cells was obtained after the LNP/Cre-GFP treatments, where the EC16-63, EC16-80, EC16-87 and EC18-63 variants exhibited more than 30% GFP-positive cells among the 12 LNPs complexes. Particularly, EC16-63-mediated (−30)GFP-Cre delivery achieved the highest delivery efficiency, with more than 50% of GFP-positive cells observed among the four LNPs. Subsequently, we further tested the gene recombination efficiency of Cre recombinase by detecting the red fluorescence DsRed generated from the HeLa-DsRed cells after 24 h incubation with the LNP/GFP-Cre complexes.

As shown in FIG. 7, limited red fluorescence signal was measured after the free (−30)GFP-Cre proteins treatment, demonstrating that the (−30)GFP-Cre alone did not induce the expression of DsRed in the cells. Compared with cells treated with free (−30)GFP-Cre without LNPs, those receiving LNP/GFP-Cre complexes expressed a more obvious red fluorescence signal, revealing that the LNPs efficiently delivered the (−30)GFP-Cre into the HeLa-DsRed cells and then initiated gene recombination functions. Among all of the lipidoids tested, the performance of EC16-63, EC16-80, EC16-87 and EC18-63-mediated (−30)GFP-Cre delivery exhibited the most enhanced genetic functionality, consistent with the intracellular efficiency shown in FIG. 6. Compared with EC16-87, EC18-63-mediated (−30)GFP-Cre delivery exhibited a slightly lower proportion of DsRed-positive cells. Taken together, three formulations of lipidoids, EC16-63, EC16-80, EC16-87 were considered as useful LNPs from the initial library, which were then adopted in follow on experiments.

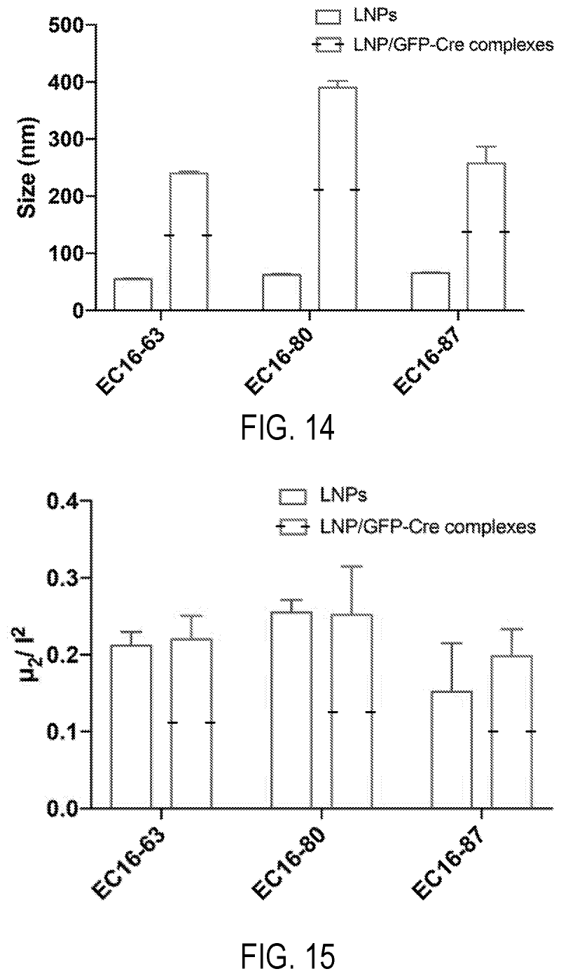

The hydrodynamic size and polydispersity index (PDI) of three LNP/GFP-Cre complexes were examined through dynamic light scattering (DLS) (FIGS. 14-15). The three LNPs exhibited comparable hydrodynamic sizes, approximately 57.2 nm in diameter, while the size dramatically increased to over 200 nm with the addition of the (−30)GFP-Cre protein. In particular, after the co-incubation with (−30)GFP-Cre protein, the size of EC16-80 increased to about 400 nm. The increased size demonstrated that the LNPs could form stable complexes with the (−30)GFP-Cre protein.

Having demonstrated that three lipidoids, EC16-63, EC16-80, and EC16-87 could efficiently deliver (−30)GFP-Cre into the cells to support gene recombination, we further detected their deliver efficiency using 2D Transwell models.

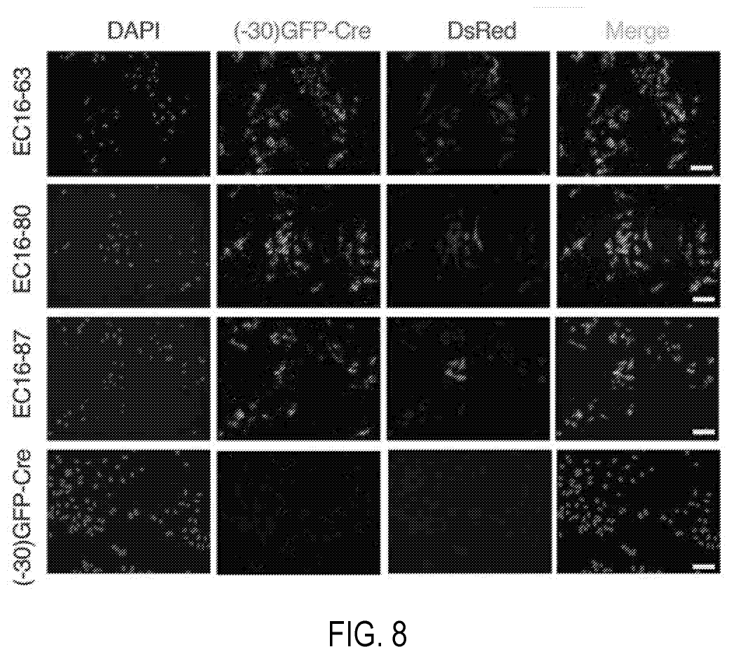

The Transwell membrane was seeded with human intestinal CaCO2/HT29-MTX cells forming a compact cell monolayer in the upper chamber with mucus-secreting features, since the mucus layer is significant in protecting the epithelial cells from damage from gut fluids (FIG. 4). Meanwhile, we also plated HeLa-DsRed cells in the bottom of the chamber to evaluate delivery efficiency of the LNPs. After incubating the LNP/GFP-Cre complexes in the upper chamber for 48 hrs, GFP-positive cells in the lower chamber were observed using fluorescence microscopy and then analyzed through flow cytometry. No fluorescence signal was observed after the addition of (−30)GFP-Cre protein into the upper chamber (FIG. 8), indicating that (−30)GFP-Cre alone did not penetrate the cell monolayer composed of the Caco2/HT29-MTX cells, which could be attributed to protein absorbance by the mucus. In contrast, green fluorescence was observed in the three LNPs-mediated (−30)GFP-Cre delivery groups, demonstrating that the LNPs loaded with the proteins could travel across the mucus layer, subsequently penetrate the cell monolayer and eventually deliver the protein into the HeLa-DsRed cells seeded at the bottom of the chamber.

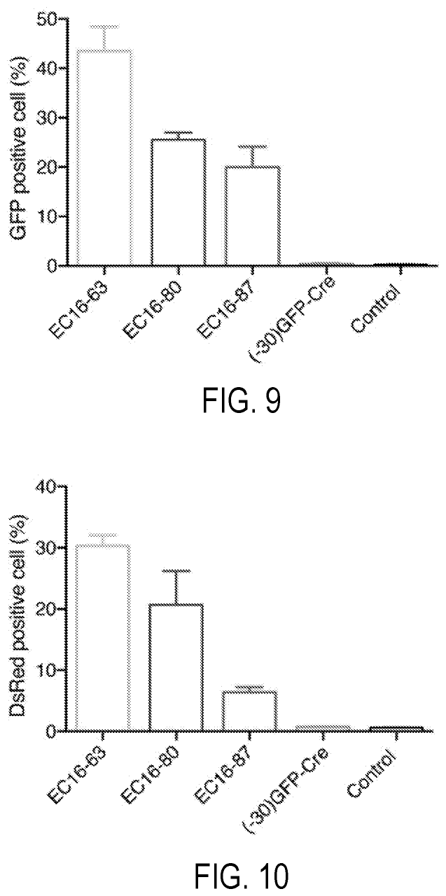

The mucus layer is composed of crosslinked and entangled mucin fibers secreted by goblet cells and submucosal glands, which exhibit negatively charged properties. Upon penetrating the mucus layer, cationic lipidoids can neutralize the negatively charged density of mucin, leading to the disruption of the crosslinks in the mucus and thereby decreasing the adhesive interactions with the nanoparticles. In addition, the addition of PEG₂ₖ in the formulation of the LNPs can enhance the stability of the nanoparticles in the mucus and accelerate the ability of the nanoparticles to cross the mucus layer. The proportion of GFP-positive cells was further quantified through flow cytometer, where EC16-63 achieved higher delivery efficiency among the three lipidoids (FIG. 9).

We also analyzed the gene recombination efficiency of complexes after (−30)GFP-Cre delivery. Once delivered into the HeLa-DsRed cells, the complexes released the (−30)GFP-Cre proteins and activated the gene-editing function to achieve the expression of intracellular red fluorescence. As shown in FIG. 10, there was no DsRed signal in the (−30)GFP-Cre protein-treated group, since the free protein could not be efficiently delivered into the cells. Meanwhile, red signal was observed in the LNPs-mediated protein delivery, indicating successful gene recombination induced by Cre protein. DsRed-positive cells were harvested and counted by flow cytometry, where it was confirmed that EC16-63 provided the most efficient gene recombination, likely due to the relatively higher delivery efficiency among the three lipidoids.

The mechanism of LNPs penetration of the 2D Transwell model was systemically investigated. First, we examined the cytotoxicity of the lipidoids by a colorimetric assay for assessing cell metabolic activity (e.g., an MTT assay, (FIGS. 14-15). The CaCO2/HT29-MTX cells were treated for 48 hrs with the same dose of LNP/GFP-Cre complexes as used in the investigation of delivery efficiency in 2D Transwell models. The lipidoids did not induce significant cytotoxicity against the CaCO2/HT29-MTX cells. Since the LNPs caused limited cytotoxicity against the cells, the transepithelial electrical resistance (TEER, EVOM2™ Epithelial Voltohmmeter) was measured in real-time to evaluate the integrity of tight junction dynamics of the monolayer.

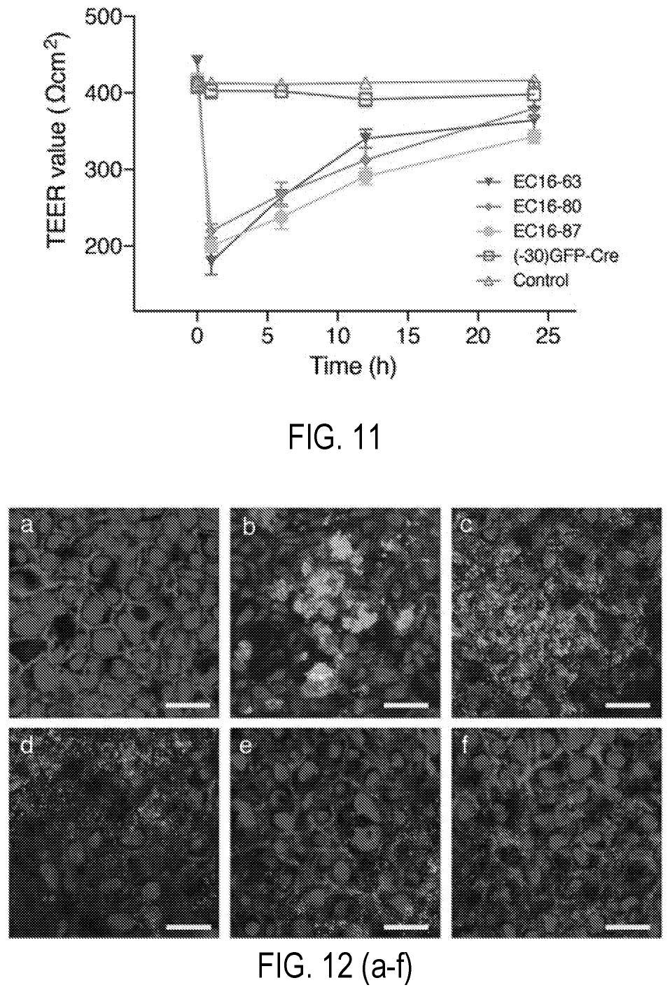

There was an significant decrease of transepithelial electrical resistance (TEER) values in the first hour after exposure to the LNPs, indicating that the complexes interrupted the tight junctions between the epithelial monolayer (FIG. 11).

A progressive recovery of the TEER values was then achieved in a time-dependent manner, illustrating that the tight junctions of the cell monolayers was gradually recovered. We also detected the tight junction changes of the cell monolayer through immunochemistry staining using EC16-63 lipidoid as a model (FIG. 12(a-f)). There was strong green fluorescence observed in LNP/GFP-Cre group in the first hour, indicating that there was an accumulation of LNPs at the surface of the epithelial cells. Moreover, the green fluorescence gradually decreased in a time-dependent manner, demonstrating that most of the LNP/GFP-Cre complexes had penetrated the cell monolayer seeded on the Transwell membranes and reached the bottom of the chamber. In contrast, after 24 hrs treatment with (−30)GFP-Cre alone, there remained an obvious green fluorescence at the surface, providing direct evidence that the (−30)GFP-Cre proteins were incapable of penetrating the cell monolayer.

Meanwhile, a reduced red fluorescence could be seen in the first hour after incubation with the LNP/GFP-Cre complexes, indicating that the tight junctions were interpreted by the cationic lipidoids. Further, after treatment for 24 hrs, the tight junctions exhibited recovery to normal levels, which was probably related to the fact that the LNPs had penetrated the cell monolayer and could not interrupt the tight junctions. Taken together, we conclude that compared with (−30)GFP-Cre alone, the LNPs-mediated protein delivery prevented (−30)GFP-Cre from adhesive interactions with the mucin, facilitating accelerated crossing the mucus layer, and penetrated the cell monolayer seeded on the Transwell membrane through the interruption of tight junction without cell damage.

Finally, based on the results with the 2D Transwell model, we investigated the performance of the LNP/GFP-Cre complexes in the 3D tissue engineered intestinal system. Compared with the 2D Transwell model, the 3D system provides more accurate assessment of the behavior of nanoparticles, since the 3D systems geometrically mimic the architecture of the human intestine, while also providing physiological conditions that more closely mimic human conditions (e.g., formation of natural oxygen gradients, higher levels of mucous formation, and interactions with gut bacteria).

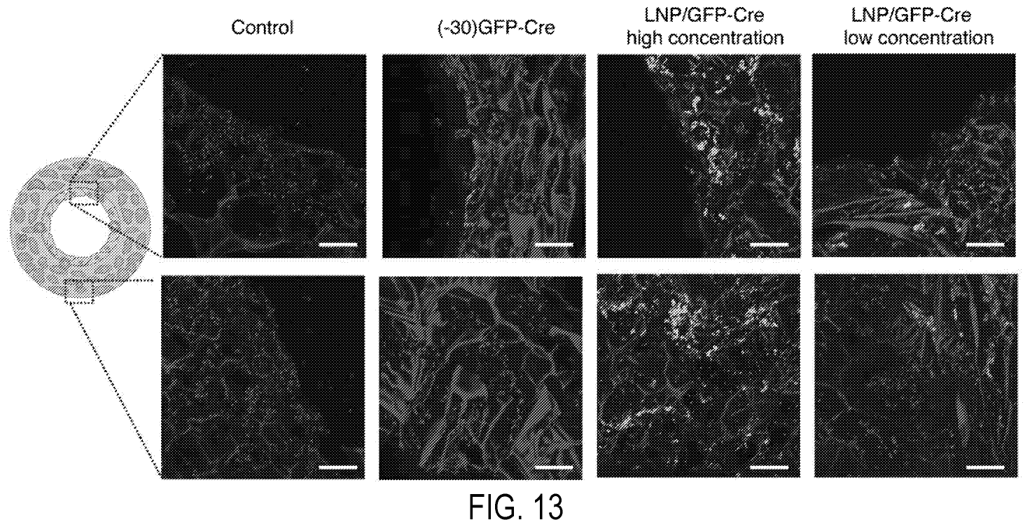

We utilized this tissue to evaluate the LNPs using EC16-63 lipidoid as models. First, the lumen surface of the 3D tissues was seeded with CaCO2/HT29-MTX cells and the bulk space was filled with HeLa-DsRed cells. Second, the 3D scaffold were perfused with LNP/GFP-Cre complexes, incubated for 48 hrs and then washed with PBS (FIG. 2). As shown in FIG. 13, green fluorescence generated from (−30)GFP-Cre was only observed at the surface of the lumen, revealing that the (−30)GFP-Cre accumulated on these surfaces but were incapable of reaching the bulk space. In contrast, after treatment with LNP/GFP-Cre complexes at different concentrations, an accumulation of green fluorescence was obtained in both the lumen and bulk space, demonstrating that the LNPs-mediated protein delivery penetrated the lumen and reach the deeper bulk space. More importantly, when (−30)GFP-Cre was delivered into the bulk space by LNPs, a red fluorescence of DsRed was detected, indicating that the (−30)GFP-Cre was functional as delivered based on intracellular gene recombination. The successful LNPs-mediated (−30)GFP-Cre protein delivery indicated that the 3D tissue models provided a useful platform for monitoring the behavior of the lipidoids nanoparticles.

In summary, we used a combinatorial library approach to synthesize a series of cationic lipidoids as a protein delivery platform. Using the (−30)GFP-Cre proteins as a model, we found that that the cationic lipidoids could protect the proteins from adhesive interactions of the mucus layer, subsequently penetrate the cell monolayer in 2D Transwell systems through the temporary interruption of tight junctions, and finally facilitate protein delivery into HeLa-DsRed cells seeded on the bottom of the chambers, achieving efficient intracellular gene recombination. Based on these 2D results with transwells, EC16-63 was selected for the 3D tissue model. After prefusion with the LNP/GFP-Cre complexes, the LNPs efficiently penetrated the cell monolayers on the surface of the lumen and reached the deeper bulk space to induce the expression of DsRed in the HeLa-DsRed cells through genetic recombination, indicating that the 3D system was beneficial for evaluating the performance of the LNPs. In summary, the combination of 2D and 3D cell and tissue culture provided a convenient platform to screen and validate potential LNPs, which were able to condense and deliver gene-editing proteins into stable nanoparticles, penetrate the intestinal cell monolayer, and maintain integrity to realize function.

The present disclosure has described one or more preferred embodiments, and it should be appreciated that many equivalents, alternatives, variations, and modifications, aside from those expressly stated, are possible and within the scope of the invention.

We claim:

1. A method for screening drug delivery vehicles for use in delivering cargo via oral delivery, the method comprising:
(i) introducing a drug delivery vehicle comprising an imaging agent into a lumen of an artificial intestine system composed of a scaffold matrix material having:
(a) an interconnected network of pores;
(b) intestinal epithelial cells positioned on an inner surface of the lumen; and
(c) human-based cells positioned within the pores and surrounding the intestinal epithelial cells;
(ii) maintaining the artificial intestine system in physiologically relevant conditions for a predetermined length of time; and
(iii) detecting a color change induced by the imaging agent within at least a portion of the human-based cells.

2. The method of claim 1, the method further comprising (iv) quantifying the number of human-based cells in the artificial intestine system that undergo the color change.

3. The method of claim 2, the method further comprising (v) repeating steps (i)-(iv) for a plurality of different drug delivery vehicles, wherein the repeating uses the artificial intestine system or different artificial intestine system for each of the plurality of different drug delivery vehicles.

4. The method of claim 3, the method further comprising (vi) generating a report that includes one or more of the following:
(a) a list ranking at least a portion of the different drug delivery vehicles based on a quantity of human-based cells in the artificial intestine system that experience the color change;
(b) a graph plotting the quantity of human-based cells in the artificial intestine system that experience the color change for at least a portion of the different drug delivery vehicles; and
(c) identification of the drug delivery vehicle with the highest quantity of human-based cells that experience the color change.

5. The method according to any one of the preceding claims, wherein the drug delivery vehicle comprises a lipid nanoparticle.

6. The method according to any one of the preceding claims, wherein the imaging agent comprises a fluorescent compound.

7. The method according to any one of the preceding claims, wherein the imaging agent comprises a gene-editing agent.

8. The method of the immediately preceding claim, wherein the gene-editing agent activates fluorescence within the human-based cells.

9. The method of any one of the preceding claims, wherein the human-based cells comprise an adenocarcinoma-based cell.

10. The method of the immediately preceding claim, wherein the adenocarcinoma-based cell is a HeLa cell.

11. The method of the immediately preceding claim, wherein the HeLa-based cell comprises a fluorescent compound that is activated by the gene-editing agent in the drug delivery vehicle.

12. The method according to any one of the preceding claims, wherein the intestinal epithelial cells comprise an adenocarcinoma-based cell.

13. The method of the immediately preceding claim, wherein the adenocarcinoma-based cell is selected from CaCO-2 cells, HT29-MTX cells, and combinations thereof.

14. The method according to claims 1-12, wherein the intestinal epithelial cells comprise at least one of: enterocytes, fibroblasts, Goblet cells, Paneth cells, and enteroendocrine cells.

15. The method according to any one of the preceding claims, wherein the scaffold matrix material is composed of a biologically-based polymer.

16. The method of the immediately preceding claim, wherein the biologically-based polymer comprises silk fibroin.

17. The method of any one of the preceding claims, wherein prior to step (i) the method includes:
introducing the drug delivery vehicle into an upper plate of a two-dimensional culture system having:
(a) a lower plate having human-based cells positioned on a surface of the lower plate;
(b) an upper plate comprising a porous membrane and intestinal epithelial cells positioned on a surface of the porous membrane, wherein the upper plate is separated from the lower plate by a distance, wherein the upper plate is spaced from the lower plate by a distance;
maintaining the two-dimensional culture system in physiologically relevant conditions for a predetermined length of time; and
detecting a color change induced by the imaging agent within at least a portion of the human-based cells.

18. The method of claim 17 further comprising quantifying the number of human-based cells in the two-dimensional culture system that undergo the color change.

19. The method of claims 17-18 further comprising repeating the steps for a plurality of drug delivery vehicles.

20. The method of claims 17-19 further comprising selecting at least a portion of the drug delivery vehicles based on the quantity of human-based cells in the two-dimensional culture system that undergo the color change, and perform steps (i)-(iii) of claim 1.

21. A method for screening drug delivery vehicles for use in delivering cargo via oral delivery, the method comprising:
introducing the drug delivery vehicle into an upper plate of a two-dimensional culture system having:
(a) a lower plate having human-based cells positioned on a surface of the lower plate;
(b) an upper plate comprising a porous membrane and intestinal epithelial cells positioned on a surface of the porous membrane, wherein the upper plate is separated from the lower plate by a distance, wherein the upper plate is spaced from the lower plate by a distance;
maintaining the two-dimensional culture system in physiologically relevant conditions for a predetermined length of time; and
detecting a color change induced by the imaging agent within at least a portion of the human-based cells.

22. A system for screening drug delivery vehicles, the system comprising:
an artificial intestine system composed of a scaffold matrix material having:
(i) an interconnected network of pores;
(ii) a lumen extending through the scaffold matrix material;
(iii) a first region of cells, the first region of cells comprising intestinal epithelial cells positioned on an inner surface of the lumen; and
(iv) a second region of cells, the second region of cells comprising human-based cells positioned within the pores and surrounding the intestinal epithelial cells.

23. The system of claim 22, wherein the first region of cells forms a monolayer of cells positioned on the inner surface of the lumen.

24. The system according to any one of the preceding claims, wherein the second region of cells express a fluorescent compound when expose to an enzyme recombinase that induces enzyme-mediated gene recombination.

25. The system of any one of the preceding claims, wherein the second region of cells completely surrounds the first region of cells.

26. The system of any one of the preceding claims, wherein the second region of cells has a thickness that is at least 1.5 times greater than the first region of cells.
